(19) United States
(12) Patent Application Publication
(10) Pub. No.: US 2005/0043272 A1
(43) Pub. Date: Feb. 24, 2005

(54) Compositions and methods for hydrophobic drug delivery

(75) Inventors: David Platt, Newton, MA (US); Eliezer Zomer, Newton, MA (US); Anatole Klyosov, Newton, MA (US)

Correspondence Address: PERKINS, SMITH & COHEN LLP, ONE BEACON STREET, 30TH FLOOR, BOSTON, MA, 02108, US

(73) Assignee: Pro-Pharmaceuticals, Inc., Newton, MA (US)

(21) Appl. No.: 10/889,555

(22) Filed: Jul. 12, 2004

Related U.S. Application Data
(60) Provisional application No. 60/486,338, filed on Jul. 11, 2003.

(51) Int. Cl. A61K 31/715

(52) U.S. Cl. 514/54

(57) ABSTRACT

Disclosed herein are compositions and methods for the delivery and targeting of therapeutics using nanometer sized polysaccharide structures. The methods and compositions described herein afford improved efficacy for pharmaceuticals such as anti-tumor drugs on metastatic tumor. The methods described herein are applicable to all chemotherapeutic agents and are especially useful for poorly soluble (hydrophobic) drugs which when formulated with the present compositions render them deliverable in physiological fluids. The methods and compositions described herein also improve the efficacy of pharmaceutical agents by targeting carbohydrate receptors specific to tumors that mediate endocytosis or enhance delivery of the drug to the ultimate site of action.

### RELATED APPLICATIONS

This application claims priority to and the benefit of U.S. Provisional Application No. 60/486,338, filed Jul. 11, 2003.

### FIELD OF THE INVENTION

The present invention relates to methods and compositions for drug delivery. In particular, the current invention pertains to methods and compositions used to deliver pharmaceutical agents especially those that have a low solubility constant in physiological fluids.

### BACKGROUND OF THE INVENTION

In attempts to increase efficacy and/or decrease toxicity, chemotherapeutic agents have been targeted to tumor cells employing so-called drug targeting techniques. Efficient drug targeting often improves the way a drug is administered. Products utilizing drug delivery technologies are generally considered novel. Control of drug concentration in the blood through the use of drug targeting improves safety and efficacy. The ultimate criterion of effective drug delivery is of course to control and optimize the targeting or increase localization of a drug at the tumor locus while concomitantly rapidly clear the non-targeted drug fraction from healthy organs/tissues.

Conventional drug delivery systems such as controlled release, sustained release, transdermal systems are based on a physical erosion process for delivering active product into the systemic circulation over time with the objective of improving patient compliance. These conventional systems do not address the biologically relevant issues such as site targeting, localized release and clearance of drug.

There are two major factors that impact the achievement of desirable drug delivery, (i) the physical characteristics of the drug that affects its interactions with the intended pharmacological target sites and undesired areas of toxicity, and (ii) the biological characteristics of the diseased area which impacts the ability of the drug to selectively interact with the intended target site to allow the drug to express the desired pharmacological activity.

Both of these factors are important in increasing efficacy and reducing toxicity of any pharmaceutical agent. Although the drug delivery industry began in response to opportunities for improved delivery of pharmaceutical compounds, efforts have primarily concentrated on making drugs compatible with physiological fluids, such as blood. Surfactants, liposomes, pegilation as well as other formulations have been used in this effort to increase drug efficacy and reduce toxicity.

There have been numerous attempts to provide a hydrophobic drug formulation, e.g., for paclitaxel, the most successful of which has been the incorporation of a drug into a liposomal formulation. However, these preparations suffer from the fact that it is difficult to achieve a predetermined drug concentration into the liposomal compartment. Moreover, the product has a short life stability.

Currently, there is a clear need for a stable, easily prepared, biocompatible, efficacious formulation for hydrophobic drugs that exhibits minimal side effects. The rationale for employing a polymeric drug carrier approach is to exploit the enhanced permeability and retention effect (EPR) by which macromolecules may accumulate and be retained at a tumor site. A second advantage of a polymeric delivery is achieving a superior pharmacokinetics (enhanced activity with altered or less severe systemic toxicity) due to longer retention in circulation and not overloading the renal or liver elimination system with that portion of the drug not bound to the polymer or retained at the tumor site. A third advantage is the direct targeting of a tumor cell's receptors thereby effectuating an increase drug concentration at the tumor locus.

### SUMMARY

The present invention pertains to methods and compositions for delivering and targeting pharmaceutical agents using one or more polysaccharide structures. The compositions and methods of the instant invention are particularly directed towards poorly soluble (hydrophobic) drugs which when formulated with one or more of the polysaccharide-based compositions of the present invention renders a hydrophobic drug deliverable in a physiological fluid. The current invention also improves therapeutic efficacy by targeting carbohydrate receptors associated with tumors. Moreover, other biologically important molecules are envisaged to be within the scope of this invention such as proteins/peptides, nucleic acids, and alike.

In one embodiment of the present invention, a polymer comprised of a polysaccharide backbone is disclosed. The polymer of the present invention can form an enclosure in which one or more small molecules, including one or more pharmaceutical agents, nucleic acids and alike can be entrapped. In a particular aspect of this embodiment, alkylated hydrocarbons are linked to the polysaccharide backbone. In one aspect, the alkylated hydrocarbon moieties attached to the polysaccharide backbone are disposed internally within the enclosure of the polymer. Hydrophobic small molecules can be sequestered within the alkylated moieties contained within the enclosure of the polymer, thereby facilitating the delivery of a hydrophobic molecule in an aqueous environment. The polysaccharide substances of the present invention can be either natural (occurring in nature) or synthetically made. These polysaccharides can be neutral such as neutral galactomannan or charged like cationic poly-glucosamine or anionic rhamnogalctan.

In another embodiment, the nano-complex of the present invention comprises target specific carbohydrates. The inclusion of these target specific carbohydrates, such as, galactose, rhamnose, mannose, or arabinose provides the surface of a polymer recognition capabilities in targeting specific lectin type receptors on the surface of cells, especially tumor cells.

As used herein, the following terms shall have the meanings indicated, unless otherwise indicated.

“Efficacy” of a therapeutic agent refers to the relationship between a minimum effective dose and a manifestation of therapeutic effects. Efficacy of an agent is increased if a therapeutic end point can be achieved by administration of a lower dose or a shorter dosage regimen; likewise, efficacy of an agent is increased if a higher therapeutic effect can be achieved by administration of a lower dose or a shorter dosage regimen. If toxicity can be decreased, a therapeutic agent can be administered on a longer dosage regimen or even chronically with greater patient compliance and improved quality of life. Further, decreased toxicity of an agent enables the practitioner to increase the dosage to achieve the therapeutic endpoint sooner, or to achieve a higher therapeutic endpoint.

The term “pharmaceutically acceptable carrier” refers to any and all solvents, dispersion media, e.g., human albumin or cross-linked gelatin polypeptides, coatings, antibacterial and antifungal agents, isotonic, e.g., sodium chloride or sodium glutamate, and absorption delaying agents, and the like that are physiologically compatible with the intended subject. The use of such media and agents for pharmaceutically active substances is well known in the art. Preferably, the carrier is suitable for oral, intravenous, intramuscular, subcutaneous, parenteral, spinal or epidural administration (e.g., by injection or infusion). Depending on the route of administration, the active compound can be coated in a material to protect the compound from the action of acids and other physiological conditions that can inactivate the pharmaceutically active compound.

“Parenteral administration” includes, but is not limited to, the administration by bolus injection or infusion, as well as administration by intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The term “toxic” as used herein means any adverse effect caused by an agent when administered to a subject.

The term “nonspecific death” refers to the death of a tumor-affected animal if its day of death was significantly different than either the control untreated animals or treated animals. “Tumor regression” was scored (excluding nonspecific deaths) as “partial” (less than fifty percent of its size of the average control untreated animal sat the beginning of treatment), or “complete” (tumor becomes unpalpable).

The term “duration of regression” refers to the interval during which a tumor classified as a partial or complete regression continues to be below 50 percent of its average size in the control untreated animals.

The term “evaluation size” refers to the tumor mass selected at one or two mass doubling beginning with the initial tumor size at the start of treatment.

“Time required for tumor mass doubling” is the time to reach the evaluation size; it is used in the calculations of the overall delay in the growth of the median tumor [(T−C)/C×100, %], where T−C (days) is the difference in the median of times postimplant for tumors of the treated (T) groups to attain an evaluation size compared to the median of the control (C) group. The T−C value is measured excluding nonspecific deaths, and any other animal that dies whose tumor failed to attain the evaluation size.

### BRIEF DESCRIPTION OF THE DRAWINGS

FIG. 1(a) depicts a polymer (b) and a cut away image of a nano-complex;

FIG. 2 depicts alkylated polysaccharides (a & b) of the present invention;

FIG. 3 is a graph illustrating the efficacy of the present invention in the treatment of cancer.

### DETAILED DESCRIPTION

The present invention pertains to methods and compositions for delivering and targeting pharmaceutical agents using one or more polysaccharide structures. In a particular embodiment, the pharmaceutical agents are anti-cancer therapeutics. The compositions and methods of the instant invention are particularly directed towards poorly soluble (hydrophobic) drugs which when formulated with one or more of the compositions of the present invention renders a hydrophobic drug deliverable in a physiological fluid. The current invention also improves therapeutic efficacy by targeting carbohydrate receptors associated with tumors. In a particular aspect, drug efficacy is improved by physical association of a drug with naturally occurring alkylated polysaccarides or chemically modified polysaccharide. The targeting aspect of the present invention is accomplished using alkylated polysaccharide containing sections or adjuncts composing for example, of galactose (e.g. galactomannans), Rhamnose (e.g. Rhamnogalactans) or mannose (e.g. Mannans).

Various types of cellular interactions mediated by cell surface components such as carbohydrate-binding proteins, called lectins, have been studied for the last twenty years. These studies have resulted in the identification of a number of compounds (such as simple sugars, and some modified polysaccharides, such as pectins) which allegedly interact with lectins on the surface of cancer cells. It has been previously reported that some tumor cell colony development was hindered when the cancer cells were treated in vitro with anti-galectin monoclonal antibodies or galactose oligomer prior to their intravenous injection into mice, as described by L. Meromsky, R. Lotan, and A. Raz, Cancer Res. 46, 5270 (1991); D. Platt and A. Raz, J. Natl. Cancer Inst. 84, 438-442 (1992). However, no such substance is currently available in clinical practice, additionally, there is a paucity of substances able to increase the efficacy of known chemotherapy drugs, such as 5-fluorouracil, doxorubicin, paclitaxell, Cis-platinum, cyclophosphamide, or others widely used in cancer chemotherapy. At best, these lectin-derived or lectin-related compounds and/or other polysaccharide-based compounds are used and/or described in the prior art as stand-alone drugs. This fact differentiates them principally from the polysaccharides disclosed herein which increase the efficacy of a known cancer chemotherapy drug being administered as admixtures with said polysaccharides.

It is generally accepted that lectins mediate many biological recognition events in plants and in animal tissues, in tumor cell lines, and cell-cell adhesion, and play a fundamental role in the organization of the extracellular matrix. Currently, lectins are defined as proteins (other than enzymes and antibodies) that have one or more binding sites for specific carbohydrate sequences, moreover, they may also display additional domains capable of interacting with molecules other than carbohydrates in nature (Barondes, S. H. TIBS 13, 480-482, 1988, the entire teaching of which is incorporated herein by reference). Lectins are diverse in structure and are characterized by their ability to bind carbohydrates with considerable specificity (Drickamer, K. Curr. Opin. Struct. Biol., 5, 612-616, 1995, the entire teaching of which is incorporated herein by reference). Animal lectins have been found associated with the cell surface, the cytoplasm, and the nucleus (Barondes, 1988, supra; Jia and Wang, J. Biol. Chem., 263, 6009-6011, 1988, the entire teaching of which is incorporated herein by reference). At the cell surface, lectins can act as receptors involved in selective intercellular adhesion and cell migration, recognition of circulating glycoproteins, and modulating cell-cell and cell-matrix interactions (Regan et al., Proc. Natl. Acad. Sci. USA 83, 2248-2252, 1986; Rosen, S. D., Curr. Opinion Cell Biol., 1, 913-919, 1989; Lehmann et al., Proc. Natl. Acad. Sci. USA 87, 6455-6459, 1990; Laing et al., J. Biol. Chem., 264, 1907-1910, 1989, the entire teaching of which is incorporated herein by reference).

Based on their protein sequence homologies, animal lectins have been classified into five distinct families (Drickamer, 1995, supra), one of these families is the galactoside binding lectins, or galectins (Raz, A, and Lotan, R., Cancer Metastasis Rev. 6, 433, 1987; Gabius, H. J., Biochem. Biophys. Acta 1071, 1, 1991, the entire teaching of which is incorporated herein by reference). Other families include C-type or Ca⁺²-dependent lectins, P-type Man 6-phosphate receptors, I-type lectins (immunoglobulin-like sugar-binding lectins), and L-type lectins (related in sequence to the leguminous plant lectins).

Galectins are members of a family of β-galactoside-binding lectins with related amino acid sequence (Barondes et al., Cell 76, 597-598, 1994; Barondes et al., J. Biol. Chem. 269, 20807-20810, 1994, the entire teaching of which is incorporated herein by reference). Currently, nine types of galectins are described in the literature. Galectin-1 is abundant in smooth and skeletal muscle, and is present in many other cell types (Couraud et al., J. Biol. Chem. 264, 1310-1316, 1989). Galectin-2 is expressed in hepatomas (Gitt et al., J. Biol. Chem. 267-10601-10606, 1992). Galectin-3 is abundant in activated macrophages and epithelial cells (Cherayil et al., Proc. Natl. Acad. Sci. USA 87, 7324-7326, 1990), and is highly expressed by oncogenically transformed and metastatic cells (U.S. Pat. No. 5,895,784). Galectin-4 is expressed in intestinal epithelium and the stomach. Galectin-4, -5, and -6 are described in Oda et al., J. Biol. Chem. 268, 5929-5939 (1993) and Barondes et al., Cell 76, 597-598 (1994). Galectin-7 is found mainly in stratified squamous epithelium (Madsen et al., J. Biol. Chem. 270, 5823-5829, 1995). Galectin-8, -9, and -10 are described in U.S. Pat. No. 6,027,916. Rat galectin-8 is most highly expressed in lung with significant expression in liver, cardiac and skeletal muscle and spleen (U.S. Pat. No. 5,869,289). While these lectins have some similarities, they are not interchangeable therapeutically or diagnostically.

Galectin-1 has been shown to either promote or inhibit cell adhesion depending upon the cell type in which it is present. It inhibits cell-matrix interactions in skeletal muscle (Cooper et al., J. Cell Biol. 115, 1437-1448, 1991, the entire teaching of which is incorporated herein by reference), promotes cell-matrix adhesion possibly by cross-linking cell surface and substrate glycoconjugates (Zhou et al., Arch. Biochem. Biophys. 300, 6-17, 1993; Skrincosky et al., Cancer Res. 53, 2667-2675, 1993, the entire teaching of which is incorporated herein by reference), participates in regulating cell proliferation (Wells et al., Cell 64, 91-97, 1991, the entire teaching of which is incorporated herein by reference) as well as some immune functions (Offner et al., J. Neuroimmunol. 28, 177-184, 1990; Perillo et al., Nature 378, 736-739, 1995, the entire teaching of which is incorporated herein by reference). Galectin-3 promotes cell growth (Yang et al., Proc. Natl. Acad. Sci. USA 93, 6737-6742, 1996, the entire teaching of which is incorporated herein by reference), its expression has been shown to be elevated in certain tumors (Raz, A and Lotan, R. Cancer Metastasis Rev. 6, 433, 1987, the entire teaching of which is incorporated herein by reference). Galectin-3, like galectin-1, has been associated with neoplastic transformation (Tinari et al., Int. J. Cancer 91, 167-172, 2001, the entire teaching of which is incorporated herein by reference). It was suggested that galectin-3 promotes tumor cell embolization in the circulatory system and enhances metastasis (Raz et al., Int. J. Cancer 46, 871-877, 1990; U.S. Pat. No. 5,895,784, the entire teaching of which is incorporated herein by reference). The function of galectin-4 remains enigmatic (U.S. Pat. No. 5,908,761, the entire teaching of which is incorporated herein by reference). Galectin-7 is thought to play a role in cell-matrix and cell-cell interactions as galectin-7 is found in areas of cell-cell contact (U.S. Pat. No. 5,869,289, the entire teaching of which is incorporated herein by reference). Galectin-8 is implicated in the regulation of cell growth, particularly in the inhibition of cell proliferation (U.S. Pat. No. 5,908,761, the entire teaching of which is incorporated herein by reference).

The present invention pertains to modified polysaccharide-based nano-suspensions. These suspensions are soluble polymers that create laminar or vesicular structures with single or multi-layer complexes. Their characteristics depend on the choice of layer components and manufacturing protocol used. For example, suspensions can be as small as 10 nanometers or as large as 2 micrometer in diameter. These suspensions can be an open unilamellar folded structure with only one compartment or multiple compartments for entrapping a drug, small molecule, nucleic acid or alike. Alternatively, suspensions can be a closed multilamellar structure with several layers capable of entrapping one or more drug molecules, small molecule, nucleic acid or alike. Moreover, the choice of the carbohydrate polymer component determines the fluidity and stability of the composition, for example, ionic and/or hydrophobic moieties can affect the flexibility/rigidity of the overall structure and affect the interaction, as well as the permeability, of a drug within the polysaccharide structure. The inclusion of target specific carbohydrates, such as, galactose, rhamnose or mannose, or arabinose provides the nano-suspension's surface recognition capabilities for targeting specific lectin type receptors on tumor cells. The polysaccharide drug complexes of the present invention affect the pharmacokinetics of a particular drug by increasing circulation time in the blood stream. Once binding to a tumor cell membrane is effectuated, the entrapped drug is released at the tumor site, alternatively, active endocytosis by a cancer cell will occur thereby facilitating the introduction of the particular drug into the cytoplasm of the cancer cell.

The types of cancer that could benefit from this invention include, but are not limited to, chronic leukemia, breast cancer, sarcoma, ovarian carcinoma, rectal cancer, throat cancer, melanoma, colon cancer, bladder cancer, lung cancer, mammary adenocarcinoma, gastrointestinal cancer, stomach cancer, prostate cancer, pancreatic cancer, or Kaposi's sarcoma. Other cancers not articulated herein but well known to those skilled in the art are also considered to be envisaged within the scope of this invention.

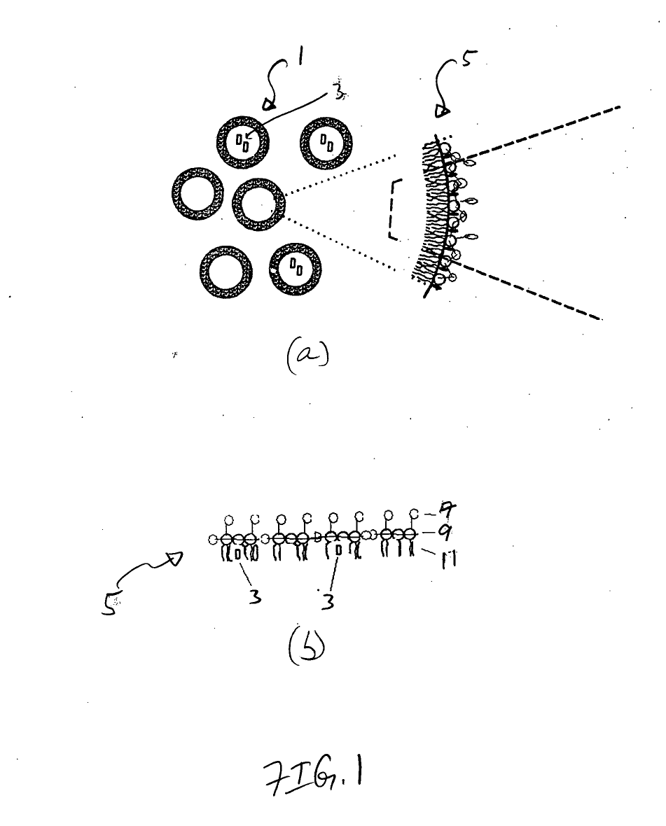

FIG. 1 depicts one embodiment of the present invention. FIG. 1a depicts a closed structure with a drug (D) 3 contained therein. The complex can range from about 10 nanometers to about 2 micrometer in diameter. A cut away of the structure is also depicted in FIG. 1a. FIG. 1b illustrates the components of the cut away depiction. The backbone 9 comprises polysaccharides linked together. The polysaccharide substrates of the present invention can be either natural (occurring in nature) or synthetically made. These polysaccharides can be neutral or charged like neutral galactomannan, cationic poly-glucosamine or anionic rhamnogalctan. Typically, the size of polysaccharide substrate used ranges from about 5 to about 1000 repeat units. A surface adduct carbohydrate ligand could be composed of a single or several of the following carbohydrates; galactose, rhamnose, mannose, fucose, sialic acid or their animated, acetylated or sulfate forms.

Polysaccharides that can be employed for the backbone include, but are not limited to, mannan, dextrans, polygalacturonate, polyglucosamine and others water soluble polysaccharides.

Also depicted in FIG. 1b are recognition moieties 7. These moieties 7 are basically carbohydrates. The inclusion of target specific carbohydrates, such as, galactose, rhamnose, mannose, or arabinose provides the polymer surface recognition capabilities in targeting specific lectin type receptors on tumor cells. Additionally, there could be a heterogenous population of carbohydrate residues on the polymer as it is the case in some natural occurring polymers, like modified pectin and some galactan. Another component depicted in FIG. 1b is the hydrophobic (alkyl) group 11. The alkyl groups 11 sequester the drugs present within the polymer.

The alkyl-polysaccharides of the present invention can originate from natural sources or by synthetic chemistry using naturally occurring carbohydrate polymers. Microbial sources for such alkylated polysaccharides are well known to those in the art, see, e.g., U.S. Pat. No. 5,997,881, the teaching of which is incorporated in its entirety by reference. Some of the microbial sources have been used in oil clean up operations, see, Gutnick and Bach “Engineering bacterial biopolymers for the biosorption of heavy metals; Applied Microbiology and Biotechnology, 54 (4) pp 451-460, (2000); also see U.S. Pat. No. 4,395,354, Gutnick , et al. 1983, the entire teaching of which are incorporated herein by reference. These microbes involved in oil clean ups have been referred to as “Emulsans” wherein some of their polysaccharides are O-acylated. Similar alkylated carbohydrates were also isolated from yeast fermentation and are known as sophorolipids.

An example of such polysaccharides is a polysaccharide chain consisting essentially of 2-amino-2,6 dideoxyaldohexose sugar, glucosamine and one or more non-aminated sugars, wherein the amine groups of the aminated sugars are substantially all, in acetylated form. The polysaccharide chain is linked with an ester bond to an alkyl moiety consisting of saturated and/or unsaturated chain of about 10 to about 18 carbon atoms of which 50-95% comprises dodecanoic acid and 3-hydroxy-dodecanoic acid. In one particular aspect, the dodecanoic acid is present in an amount greater than the 3-hydroxy-dodecanoic acid.

Optionally, the alkylated polysaccharide can comprise anionic groups, such as phosphate, sulfate, nitrate, carboxyl groups, and/or sulfate groups, while maintaining the hydrophobic moieties. The nano-suspension can be composed of one or more polymers or copolymers of the present invention.

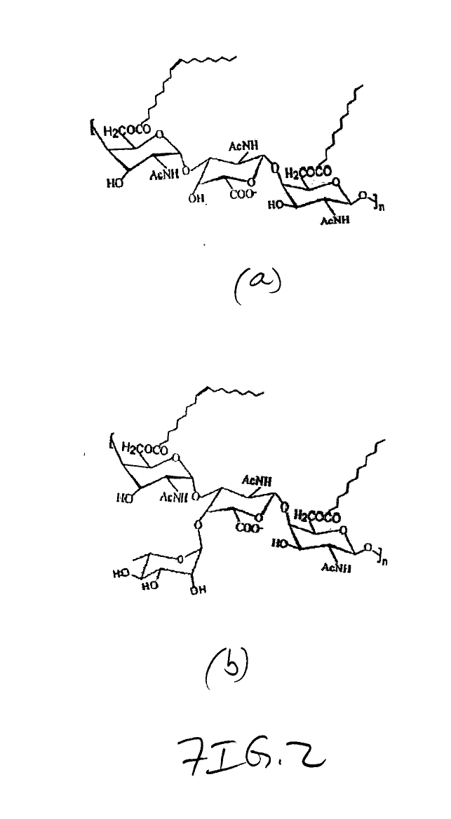

In one embodiment, a synthetic polysaccharide forms part of the nano-suspension and is esterified with straight or branched alkyl groups of about 8 to about 40 carbon atoms. These alkyl groups may be aliphatic or unsaturated, and optionally may contain one or more aromatic groups. In one embodiment, the surface of the alkylated polysaccharides of the present invention can be further derivitized using carbohydrate ligands, e.g. galactose, rhamnose, mannose or arabinose, to further enhance recognition sites by lectins on cancer surface. See FIG. 2. The polysaccharides of the present invention can be derivitized using alkyl, aryl or other chemical moieties. In a particular aspect, the derivitizing moiety is selected so that it will reversibly interact with the pharmaceutical used. This reversible interaction includes hydrophobic interactions, hydrogen bonding, ionic interactions as well as other interactions between molecules. The hydrophobic moiety includes, but not limited to, both alkyls and aryls such as decyl-, octyl-, octydecyl-, benzyl-, and phenyl-derived polymannose, polygalactose, galactomannan, Rhamnogalactan or other carbohydrate based oligomers.

To illustrate the current invention, a typical formulation is described hereinbelow using paclitaxel as the therapeutic agent. Drugs other than paclitaxel can be used such as daunomycin, doxorubicin, vinblastine, bleomycin, baccatin III, and virtually any other pharmaceutical agent (or small molecule). Even though this technology is most desirable for hydrophobic drugs, hydrophilic drugs can equally be employed. A composition for the intravenous administration of paclitaxel in a stable carbohydrate microdispersion was prepared in physiological saline using approximately 10 to about 30 mg of paclitaxel dissolved in about 100 to about 300 mL of ethanol. Other organic solvents can be employed as long as they are non-toxic to the subject. Than, approximately 10 volumes Of 10 to about 300 mg/mL of alkyl galactogalcturonic solution was added to the organic solution forming a final concentration of about 10 mg/mL and then vigorously mixed. The suspension created was then sonicated for approximately 120 seconds at 600 watts output, and 20 kHz converter, in order to create a micro-suspension with a particular size range from less than 0.1 to about 5 μm. The suspension was further processed using a microfluidizer set at approximately 18,000 psi in order to provide an emulsion type suspension with a particle size in the nanometer range. The preparation was then ready for pasteurization and administration into a suitable subject via means well known by those skilled in the art, such as intravenous injection.

In another embodiment, the carbohydrate nano-suspension of the present invention can be employed to deliver hydrophobic peptide/protein biologics. These peptide/protein molecules are sequestered within the carbohydrate polymer and delivered to the subject. Surface carbohydrates can facilitate specific interaction with a targeted cell.

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety which are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

In another embodiment, minor amounts of additives well known in the pharmaceutical field such as substances that enhance isotonicity and chemical stability can be added. Such materials are non-toxic to recipients at the dosages and concentrations envisaged (i.e., that which is suitable for the recipient) and include buffers such as phosphate, citrate, succinate, acetic acid, and other acceptable acids or their salts; antioxidants such as ascorbic acid; low molecular weight (less than about ten residues) polypeptides, e.g., polyarginine or tripeptides;; amino acids, such as glycine, glutamic acid, aspartic acid, or arginine; monosaccharides, disaccharides, and other carbohydrates including glucose, manose, or dextrins; chelating agents such as EDTA; sugar alcohols such as mannitol or sorbitol; counterions such as sodium; and/or nonionic surfactants such as polysorbates, poloxamers, or PEG.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodi-fluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with optionally an added preservative. The compositions can take such forms as suspensions, solutions or emulsions in aqueous vehicles, and can contain formulatory agents such as suspending, stabilizing and/or dispersing agents.

Pharmaceutical formulations for parenteral administration include aqueous solutions of the active compounds in water-soluble form. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Alternatively, the compounds can be delivered using a sustained-release system, such as semipermeable matrices of solid hydrophobic polymers containing the therapeutic agent. Various of sustained-release materials have been established and are well known to those skilled in the art. Sustained-release capsules can, depending on their chemical nature, release the compounds for a few days up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

### EXAMPLES

### Example A

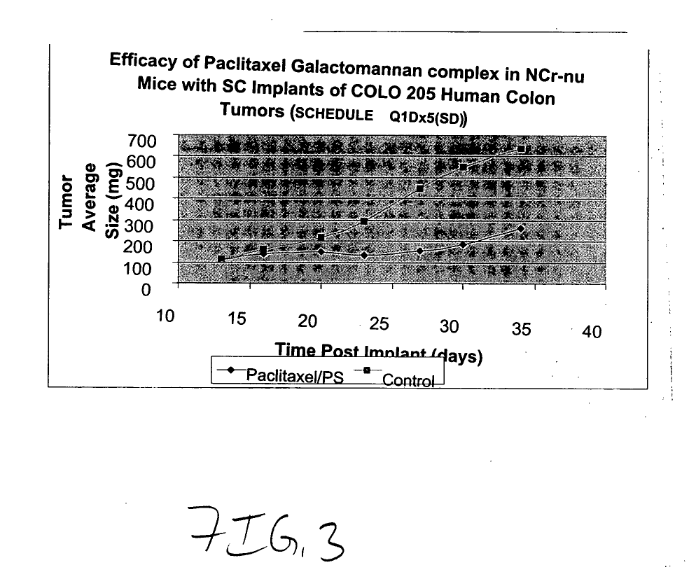

In vivo study of COLO 205 human colon cancer: The response of a subcutaneously implanted COLO 205 human colon tumor to treatment using the cytotoxic chemotherapeutic agent paclitaxel in combination with a modified galactomannan was evaluated in male NCr-nu athymic nude mice. See FIG. 3.

Male NCr-nu athymic nude mice (Frederick Cancer Research and Development Center, Frederick, Md.) were acclimated in the laboratory one week prior to the experimentation. The animals were housed in microisolator cages, five per cage, in a 12-hour light/dark cycle. The animals received filtered water and sterile rodent food ad libitum. The animals were observed daily and clinical signs were noted. Weight of the animals ranged from 25-34 g at the 13th day of the study, i.e., the first day of treatment initiation. The mice were healthy and not previously used in other experimental procedures.

Thirty to forty milligram specimens of COLO 205 human colon tumor were implanted subcutaneously (s.c.) in the mice near the right axillary area using a 12-gauge trocar needle and allowed to grow. Tumors reached between 75-198 mg in weight (75-198 mm³ in size) before the start of treatment. A sufficient number of mice were implanted so that tumors in a weight range as narrow as possible were selected for the trial on the day of treatment initiation (day 13 post tumor implantation). Those animals selected with tumors in the proper size range were divided into the various treatment groups. The median tumor weights in each treatment group ranged from 94 to 117 mg.

Study duration was 2 months, the s.c. tumors were measured and the animals were weighed twice weekly starting with the first day of treatment. Tumor volume was determined by caliper measurements (mm) and using the formula for an ellipsoid sphere: L×W2/2=mm³, where L and W refer to the larger and smaller dimensions collected at each measurement. This formula was also used to calculate tumor weight, assuming unit density (1 mm³=1 mg).

A paclitaxel/modified galactomannan (6 mg/kg/60 mg/kg) complex was administered intravenously (i.v.) with the following schedule Q1D×5 (SD). While control untreated tumors grew well in all mice and reach about 600 mg in 30 days, the tumor in treated mice reach less than 200 mg in 30 days, a 200% reduction in tumor size vs. untreated control animals.

### Example B

In vitro study of HT-29 human colon cancer: The assay was conducted using a 96 well plate formate. Paclitaxel (Sigma, US) was dissolved first in ethanol at 10 mg/mL solution and then emulsified at the ratio of 1 to 9 using a 10 mg/mL solution of modified alkyl Galactgalactan (purified from crude powder prepared from the fermentation broth of Acinetobacter calcoaceticus (PETROFERM, INC, FL.). The suspension was serially diluted in saline and added to growth media in a 96 wells plate. Each vial was inoculated with HT-29 human tumor cell suspension (approximately 1000 to 10000 cells/well). Incubation at 37° C. for 48 to 72 hours was done and results were observed at optical density of 490 nm. Control wells (no drugs gave readings at about 1.500 Optical Density units while 100% inhibition gave reading at 0.500 Optical units (using as positive control h-TNF (tumor necrosis factor) at 0.01 microgram/mL). The LD50 was calculated at less than 10 nanogram paclitaxel per mL. With 100% cytotoxic effect at 50 to 100 nanogram per mL.

While this invention has been particularly shown and described with references to specific embodiments, it will be understood by those skilled in the art that various changes in form and details may be made therein without departing from the spirit and scope of the invention as defined by the appended claims.

1. A pharmaceutical composition comprising a polymer and one or more small molecules, wherein said polymer has a polysaccharide backbone, and wherein one or more hydrophobic hydrocarbon moieties are linked to said polysaccharide backbone.

2. The composition of claim 1, wherein said polymer forms an enclosure, wherein said hydrophobic hydrocarbon moieties are disposed internally with said enclosure.

3. The composition of claim 2, wherein said small molecules have hydrophobic moieties.

4. The composition of claim 1, wherein said hydrophobic hydrocarbon moieties are selected from the group consisting of alkyl, aryl moieties, and a combination thereof.

5. The composition of claim 4, wherein said hydrocarbon hydrophobic moieties are selected from the group consisting of decyl, octyl, octydecyl, benzyl, phenyl groups, and a combination thereof.

6. The composition of claim 1, wherein said polysaccharide is natural or synthetically manufactured.

7. The composition of claim 6, wherein said polysaccharide comprises sugar moieties selected from the group consisting of neutral, anionic, cationic carbohydrate residues or a combination thereof.

8. The composition of claim 7, wherein said polysaccharide comprises galactomannan.

9. The composition of claim 7, wherein said polysaccharide comprises rhamnogalactan.

10. The composition of claim 7, wherein said polysaccharide comprises carbohydrate residues selected from the group consisting of galactose, rhamnose, mannose, arabinose or a combination thereof.

11. The composition of claim 1, wherein said small molecules are selected from the group consisting of pharmaceutical agents, nucleic acids, peptides, and a combination thereof.

12. The composition of claim 11, wherein said pharmaceutical agents comprise one or more oncolytic agents.

13. The composition of claim 12, wherein said oncolytic agents are selected from the group consisting of 5-fluorouricil, doxorubicin, paclitaxil, Cis-platinum, cyclophosphamide, daunomycin, vinblastine, bleomycin, baccatin III, and combinations thereof.

14. The composition of claim 1 having a size ranging from about 10 nm to about 2 μm.

15. The composition of claim 1 further comprises a target specific carbohydrate.

16. The composition of claim 15, wherein said target specific carbohydrate is selected from the group consisting of galactose, rhamnose, mannose, arabinose or a combination thereof.

17. The composition of claim 15, wherein said target specific carbohydrate interacts with one or more lectin-type receptors of a target cell.

18. A pharmaceutical composition comprising a polysaccharide backbone that forms an enclosure, wherein said polysaccharide backbone has one or more hydrophobic hydrocarbon moieties, and wherein said hydrophobic hydrocarbon moieties is disposed within an interior pocket of said polysaccharide enclosure, and one or more small molecules.

19. The composition of claim 18, wherein said polysaccharide comprises sugar moieties selected from the group consisting of neutral, anionic, cationic carbohydrate residues or a combination thereof.

20. The composition of claim 19, wherein said polysaccharide comprises galactomannan.

21. The composition of claim 19, wherein said polysaccharide comprises rhamnogalactan.

22. The composition of claim 19, wherein said polysaccharide comprises carbohydrate residues selected from the group consisting of galactose, rhamnose, mannose, arabinose or a combination thereof.

23. The composition of claim 18, wherein said hydrophobic hydrocarbon moieties are selected from the group consisting of alkyl, aryl moieties, and a combination thereof.

24. The composition of claim 23, wherein said hydrocarbon hydrophobic moieties are selected from the group consisting of decyl, octyl, octydecyl, benzyl, phenyl groups, and a combination thereof.

25. The composition of claim 18, wherein said small molecules are selected from the group consisting of pharmaceutical agents, nucleic acids, peptides, and a combination thereof.

26. The composition of claim 25, wherein said pharmaceutical agents comprise one or more oncolytic agents.

27. The composition of claim 26, wherein said oncolytic agents are selected from the group consisting of 5-fluorouricil, doxorubicin, paclitaxil, Cis-platinum, cyclophosphamide, daunomycin, vinblastine, bleomycin, baccatin III and combinations thereof.

28. The composition of claim 18 having a size ranging from about 10 nm to about 2 μm.

29. The composition of claim 18 further comprises a target specific carbohydrate.

30. The composition of claim 29, wherein said target specific carbohydrate is selected from the group consisting of galactose, rhamnose, mannose, arabinose or a combination thereof.

31. The composition of claim 29, wherein said target specific carbohydrate interacts with one or more lectin-type receptors of a target cell.

32. A method of delivering a small molecule to a subject in need thereof, comprising
(a) obtaining a pharmaceutical composition having a polymer and one or more small molecules, wherein said polymer has a polysaccharide backbone, and wherein one or more hydrophobic hydrocarbon moieties are linked to said polysaccharide backbone; and
(b) administering an effective amount of (a) to said subject in need thereof.

33. The method of claim 32, wherein said polymer forms an enclosure, wherein said hydrophobic hydrocarbon moieties are disposed internally with said enclosure.

34. The method of claim 33, wherein said small molecules have hydrophobic moieties.

35. The method of claim 32, wherein said hydrophobic hydrocarbon moieties are selected from the group consisting of alkyl, aryl moieties, and a combination thereof.

36. The method of claim 35, wherein said hydrocarbon hydrophobic moieties are selected from the group consisting of decyl, octyl, octydecyl, benzyl, phenyl groups, and a combination thereof.

37. The method of claim 32, wherein said polysaccharide is natural or synthetically manufactured.

38. The method of claim 37, wherein said polysaccharide comprises sugar moieties selected from the group consisting of neutral, anionic, cationic carbohydrate residues or a combination thereof.

39. The method of claim 37, wherein said polysaccharide comprises galactomannan.

40. The method of claim 37, wherein said polysaccharide comprises rhamnogalactan.

41. The method of claim 37, wherein said polysaccharide comprises carbohydrate residues selected from the group consisting of galactose, rhamnose, mannose, arabinose or a combination thereof.

42. The method of claim 32, wherein said small molecules are selected from the group consisting of pharmaceutical agents, nucleic acids, peptides, and a combination thereof.

43. The method of claim 42, wherein said pharmaceutical agents comprise one or more oncolytic agents.

44. The method of claim 43, wherein said oncolytic agents are selected from the group consisting of 5-fluorouricil, doxorubicin, paclitaxil, Cis-platinum, cyclophosphamide, daunomycin, vinblastine, bleomycin, baccatin III, and combinations thereof.

45. The method of claim 32 having a size ranging from about 10 nm to about 2 μm.

46. The method of claim 32 further comprises a target specific carbohydrate.

47. The method of claim 46, wherein said target specific carbohydrate is selected from the group consisting of galactose, rhamnose, mannose, arabinose or a combination thereof.

48. The method of claim 46, wherein said target specific carbohydrate interacts with one or more lectin-type receptors of a target cell.
